(19) United States
(12) Patent Application Publication
(10) Pub. No.: US 2022/0071578 A1
(43) Pub. Date: Mar. 10, 2022

(54) IMPROVED METHOD OF ACQUIRING A RADIOGRAPHIC SCAN OF A REGION-OF-INTEREST IN A METAL CONTAINING OBJECT

(71) Applicant: CARESTREAM DENTAL LLC, Atlanta, GA (US)

(72) Inventors: Jay S. SCHILDKRAUT, Rochester, NY (US); Jean-Marc INGLESE, Bussy-Saint-Georges (FR); Victor C. WONG, Rochester, NY (US)

(21) Appl. No.: 17/419,094

(22) PCT Filed: Dec. 30, 2019
(86) PCT No.: PCT/US2019/068893

§ 371 Date: Jun. 28, 2021

Related U.S. Application Data
(60) Provisional application No. 62/786,467, filed on Dec. 30, 2018.

(51) Int. Cl. A61B 6/00; A61B 6/03

(52) U.S. Cl. CPC A61B 6/4085; A61B 6/469; A61B 6/488; A61B 6/032

(57) ABSTRACT

The present disclosure describes a Cone Beam Computed Tomography (CBCT) imaging system and methods of operating the system to minimize the degradation of projection images by metal in a scanned object. The methods determine the location of metal in the scanned object by making an initial low dose scan and then, using information obtained from the low dose scan, perform a second scan that may be used to create a reconstruction with reduced artifacts. The methods also calculate X-ray source and detector scan trajectories which minimize reconstruction artifacts and optimize image quality, especially when a region-of-interest is near metal in the scanned object. Additionally, the methods of the present invention calculate X-ray source and detector scan trajectories that maximize the angular range of X-rays which pass through the region-of-interest that are not blocked by metal in the scanned object.

### FIELD OF THE INVENTION

The present invention relates generally to the field of methods and apparatuses for Cone Beam Computed Tomography (CBCT) imaging. More specifically, the invention relates to methods and apparatuses for improving CBCT results by optimally scanning a region-of-interest in an object which contains metal.

### BACKGROUND OF THE INVENTION

Three-dimensional (3-D) volume imaging can be a valuable diagnostic tool that offers significant advantages over earlier two-dimensional (2-D) radiographic imaging techniques for evaluating the condition of a patient's teeth and bone. Three-dimensional (3-D) imaging of a patient or other subject has been made possible by a number of advancements, including the development of high-speed imaging detectors, such as digital radiography (DR) detectors that enable multiple images to be taken in rapid succession.

Cone beam computed tomography (CBCT) (also sometimes referred to herein as cone beam CT) technology offers considerable promise as one type of diagnostic tool for providing three-dimensional (3-D) volume images. Cone beam X-ray scanners are used to produce three-dimensional (3-D) images of dental patients for the purposes of diagnosis, treatment planning, restorations, and other purposes. Cone beam CT systems capture volume data sets by using a high frame rate, flat panel digital radiography (DR) detector and an X-ray source, typically affixed to a gantry that revolves about the subject to be imaged. The cone beam CT system directs, from various points along its orbit around the subject, a divergent cone beam of X-rays through the subject and to the detector. The cone beam CT system captures projection images throughout the source-detector orbit, for example, with one two-dimensional (2-D) projection image at every degree increment of rotation. The projections are then reconstructed into a three-dimensional (3-D) volume image using various methods. Among the most common methods for reconstructing a three-dimensional (3-D) volume image from two-dimensional (2-D) projections are filtered back projection (FBP) and Feldkamp-Davis-Kress (FDK) methods.

Although three-dimensional (3-D) images of diagnostic quality can be generated using CBCT systems and technology, a number of technical challenges remain. Highly dense objects, such as metallic implants, appliances, surgical clips and staples, dental fillings, and the like can cause various image artifacts that can obscure useful information about the imaged tissue. Dense objects, having a high atomic number, attenuate X-rays in the diagnostic energy range much more strongly than do soft tissue or bone features, so that far fewer photons reach the imaging detector through these objects. For three-dimensional (3-D) imaging, the image artifacts that can be generated by metallic and other highly dense objects include dark and bright streaks that spread across the entire reconstructed image. Such artifacts can be due to physical effects such as high noise, photon starvation, radiation scatter, beam hardening, the exponential edge-gradient effect, aliasing, and clipping, and non-linear amplification in FBP or other reconstruction methods. The image degradation commonly takes the form of light and dark streaks in soft tissue and dark bands around and between highly attenuating objects. These image degradations are commonly referred to as artifacts because they are a result of the image reconstruction process and only exist in the image, not in the scanned object. These artifacts not only conceal the true content of the object, but can be mistaken for structures in the object. Artifacts of this type can reduce image quality by masking other structures, not only in the immediate vicinity of the dense object, but also throughout the entire image. At worst, this can falsify computed tomography (CT) values and even make it difficult or impossible to use the reconstructed images effectively in assessing patient conditions or for planning suitable treatments.

Dental volume imaging can be particularly challenging because of the relative complexity of structures and shapes and because objects of very different densities are closely packed together in a relatively small space. Various types of fillings, implants, crowns, and prosthetic devices of different materials can be encountered during an imaging scan. Beam hardening effects can also impact image quality. Thus, metal artifacts reduction can be particularly difficult for dental volume imaging.

The reduction of artifacts that are caused by metal and other highly attenuating objects is an essential part of a dental cone beam scanner, particularly with the increasing use of implants in dental treatments. While metal artifact reduction (MAR) reconstruction methods have been developed which are very effective, it is preferable to acquire projection images which are minimally degraded by the presence of metal in the object. Originally, cone beam CT scanners were only capable of moving the X-ray source and detector in a circle around an isocenter (center-of-rotation), however next generation scanners are able to change the location of the center-of-rotation during the scan. This has the advantage of increasing the size of the reconstructed volume and can also be used to capture projections which are minimally degraded by metal objects.

Therefore, there is a need in the industry for methods and apparatuses that solve these and other problems, difficulties, and shortcomings with current technology.

### SUMMARY OF THE INVENTION

Broadly described, the present invention comprises a Cone Beam Computed Tomography (CBCT) imaging system having a movable gantry center-of-rotation and methods for capturing projection images with the system that minimize degradation of the projection images by metal in the scanned object. According to example embodiments of the present invention, the methods determine the location of metal in the scanned object by making an initial low dose scan and then using information obtained from the low dose scan to perform a second scan that may be used to create a reconstruction with reduced artifacts. Also, a reconstruction is created that optimally images a region-of-interest which is automatically determined or indicated by a user of the scanner. Additionally, methods of the present invention calculate X-ray source and detector scan trajectories which minimize reconstruction artifacts and optimize image quality, especially when the region-of-interest is near the metal in the scanned object. In addition, methods of the present invention calculate X-ray source and detector scan trajectories that maximize the angular range of X-rays which pass through a region-of-interest (ROI) that are not blocked by metal in the scanned object.

Other features and advantages of the present invention will become apparent from reading the following description of the non-limiting, example embodiments with reference to the appended drawings.

### BRIEF DESCRIPTION OF THE DRAWINGS

FIG. 1 displays a schematic view of a Cone Beam Computed Tomography (CBCT) imaging system with a movable gantry center-of-rotation in accordance with the example embodiments of the present invention.

FIG. 2 displays a schematic view of a Cone Beam Computed Tomography (CBCT) imaging system with a moving center-of-rotation and demonstrates a method of using the system, in accordance with the example embodiments of the present invention, to improve the image quality of a feature of interest inside of a patient's head.

FIG. 3 displays a flowchart representation of a method of operating the CBCT system of FIG. 1 to improve the image quality of a region-of-interest in a radiographic scan of a metal containing object in accordance with the example embodiments of the present invention.

### DETAILED DESCRIPTION OF THE EXAMPLE EMBODIMENTS

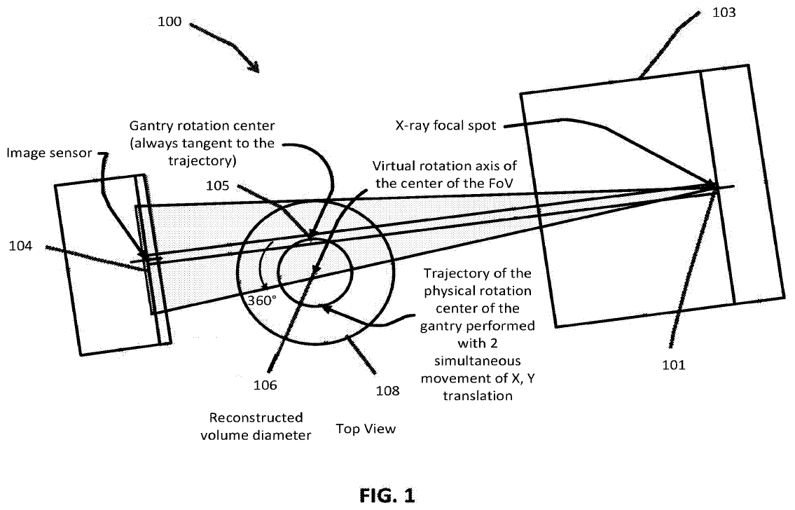

Example embodiments of the present invention are described below in detail with reference being made to the drawings in which like numerals identify like elements or steps throughout the several views. FIG. 1 displays a Cone Beam Computed Tomography (CBCT) imaging system 100 (sometimes referred to herein as the “system 100”) with a movable gantry center-of-rotation. A focal spot 101 of an X-ray source 103 emits X-rays which are detected by image sensor 104. The center of gantry rotation 105 moves in a circle about location 106. The CBCT imaging system 100 has the advantage of allowing for an increased reconstructed volume diameter 108 than would be possible for a specific detector without displacing the detector.

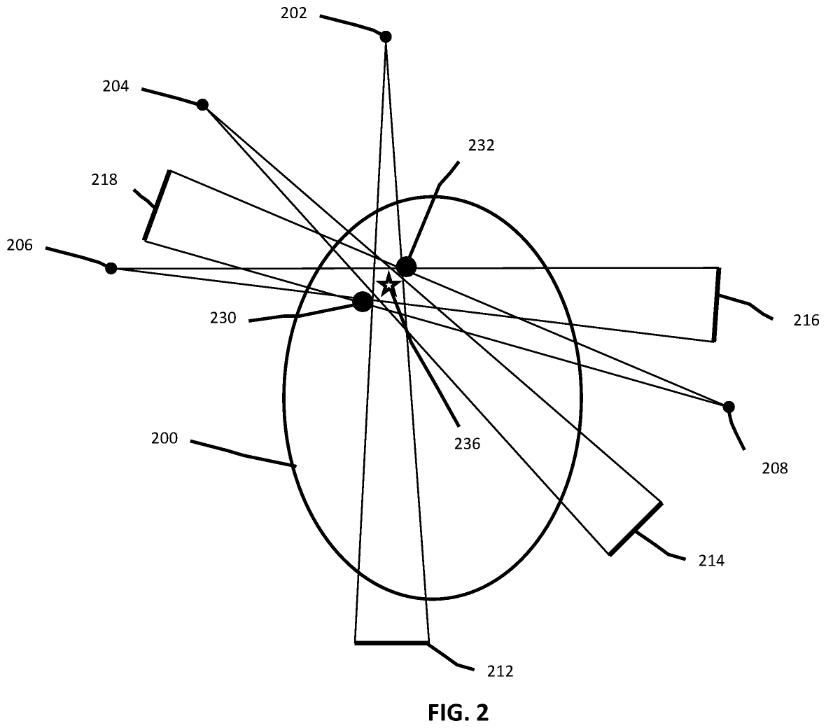

FIG. 2 displays a schematic view of the CBCT imaging system 100 with a moving gantry center-of-rotation and demonstrates a method of using the system 100 to improve the image quality of a feature of interest 236 (also referred to herein as “feature 236” or a “region-of-interest 236”) inside of a patient's head 200. Feature 236 is initially blocked by metal objects 230, 232. By moving the gantry center-of-rotation, projections are captured at exemplary source locations 202, 204, 206, and 208 with corresponding detector locations 212, 214, 216, and 218, respectively. As illustrated in FIG. 2, the gantry center-of-rotation can be modified throughout a scan to increase the range of source-detector locations that capture a projection image of feature 236 with X-rays which are not blocked metal 230, 232. Source-detector locations which substantially include X-rays that are blocked by metal 230, 232 are avoided during scanning with the CBCT system because these X-rays add dose to the patient without contributing to the image content in the reconstructed volume.

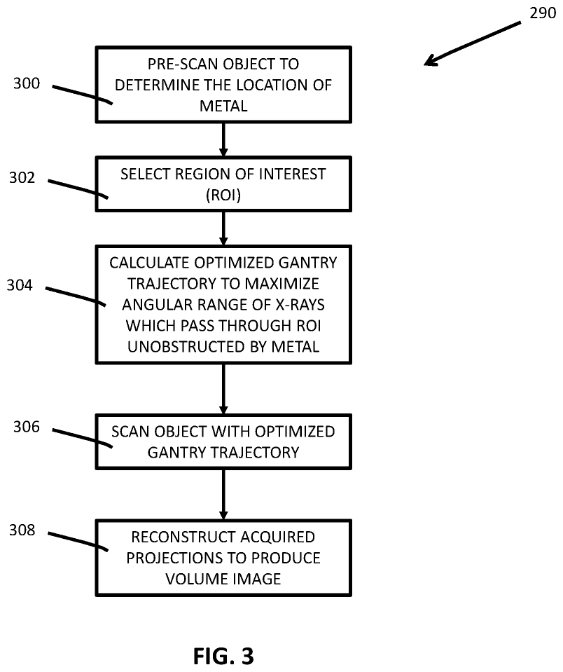

FIG. 3 displays a flowchart representation of a method 290 of operating the CBCT system 100 to improve the image quality of a region-of-interest 236. At step 300, a low dose pre-scan of the object or patient is performed which determines the location of metal. Then, at step 302, a region-of-interest (ROI) 236 is selected either by the CBCT system user or by a method which detects the location of a region-of-interest 236 that needs to be optimally imaged. Metal objects in dental and orthopedic applications are often implants and the region-of-interest 236 is often the tissue which is bordering on these implants. Next, at step 304, a gantry trajectory is calculated that maximizes the angular range of X-rays which pass through the region-of-interest 236 and are not blocked by the metal. Subsequently, at step 306, the object is scanned using this calculated optimal trajectory. Continuing at step 308, the acquired projections are reconstructed to form a three-dimensional (3-D) volume image which provides an optimal image of the region-of-interest 236. In the reconstruction process, the projection pixels are weighted to take into account the redundancies of the captured X-rays.

It should be understood and appreciated that while the present invention has been described above in connection with an example embodiment in which the CBCT imaging system 100 includes a rigidly connected X-ray source and detector, the source and detector may be moved and oriented independently in other example embodiments that are within the scope of the present invention. Also, it should be understood and appreciated that the motion of the X-ray source and detector are not confined to a plane, but includes movement and orientation in three spatial dimensions. Additionally, it should be understood and appreciated that while the present invention has been described herein with respect to the above example embodiments, the present invention may be embodied in other example embodiments that include variations from the above-described methods and apparatuses that are still within the scope of the present invention.

What is claimed is:

1. A method for acquiring a volume radiographic image of a metal containing object comprising the steps of:
acquiring an initial scan of the object;
using the initial scan to determine the location of metal in the object;
determining a region-of-interest in the object;
calculating an X-ray source and detector trajectory which maximizes the angular range of X-rays which pass through the region-of-interest unobstructed by metal;
acquiring projection images of the object at various locations along the trajectory; and
reconstructing the acquired projections to form a volume radiographic image.

2. The method of claim 1, wherein the step of acquiring includes acquiring an initial scan of the object using a low dose of X-rays.

3. The method of claim 1, wherein the step of determining includes receiving user input identifying the region-of-interest.

4. The method of claim 1, wherein the step of determining includes detecting the location of the region-of-interest that needs to be optimally imaged.

5. The method of claim 1, wherein the step of reconstructing includes weighting projection pixels to account for redundancies of the captured X-rays.

6. The method of claim 1, wherein the step of acquiring includes moving an X-ray source and detector in unison along the trajectory.

7. The method of claim 1, wherein the step of acquiring includes moving an X-ray source and detector independently along the trajectory.

8. The method of claim 1, wherein the initial scan comprises a first scan of the object and step of acquiring comprises a second scan of the object.
